(19) United States
(12) Patent Application Publication
(10) Pub. No.: US 2009/0227875 A1
(43) Pub. Date: Sep. 10, 2009

(54) Three-dimensional Imaging System

(75) Inventors: Densen Cao, Sandy, UT (US); Zhao-hui Lin, Salt Lake City, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO GROUP, INC., West Jordan, UT (US)

(21) Appl. No.: 12/397,266

(22) Filed: Mar. 3, 2009

Related U.S. Application Data
(60) Provisional application No. 61/033,652, filed on Mar. 4, 2008.

(51) Int. Cl. A61B 6/00

(52) U.S. Cl. 600/476

(57) ABSTRACT

The present invention is a three-dimensional scanner for use in the oral cavity. The scanner of the present invention utilizes three optical scanners in a single-hand wand to create three-dimensional images with a single pass of the wand over the area scanned. Processing of the collected images is then composited into a three-dimensional image for CAD/CAM restoration and diagnostic aid in dentistry. OCT technology may be utilized to generate the images.

### FIELD OF THE INVENTION

The present invention relates to the field of dentistry and more particularly relates to a three-dimensional tomography system for imaging and diagnosing the oral cavity features contained within.

### BACKGROUND OF THE INVENTION

Three-dimensional image systems including image scanning, image computer processing, and machining of processed image have been widely used in dentistry and other fields. The key area is the imaging scanning process. Several products like CEREC™ by Sirona, E4D™ for Dentist and E4D™ for Laboratory by D4D Tech, ETREO™ by Cadent, Lava™ by 3M have used a laser scanning method or CCD to catch the image of the surface scanned and use proprietary algorithms to build three-dimensional images. Such systems require multiple scans in different directions in order to build three-dimensional images. The surface conditions are also highly restricted to obtain high quality images. These systems, then, can only generate three-dimensional images and do not have any diagnostic function.

This invention is to use an optical technology in a three dimensional head to obtain three-dimensional images in the oral environment with one scan. The image will not only display the physical information of three-dimensional surface of the tooth, but also the integrity of tooth surface. This invention can also employ optical coherence tomography (OCT) technology in the system to generate images for diagnostics.

### SUMMARY OF THE INVENTION

In view of the foregoing disadvantages inherent in the known types of tomography scanners, this invention provides a three-dimensional tomography scanner. As such, the present invention's general purpose is to provide a new and improved three-dimensional scanner that provides three dimensional and diagnostic images with a single scan. To accomplish these objectives, the scanner comprises three optical scanning heads, each consisting with a micro electromechanical system (MEMS) like an endoscope probe used in medicine, which are set up at perpendicular directions (or special fixed directions with known relative angles) in order to cover the whole of a tooth. The system can also use OCT technologies to generate images for diagnostics. The system uses stereo matching to establish correspondence between the three images using proprietary methods, and a three dimensional image is thereby formed.

The system is intended to take dental restoration to a high level of productivity, patient comfort, and convenience with its 3D CAD/CAM restorative system for dental offices and laboratories. The system can produce digital 3D impressions of teeth for a variety of needs including diagnostics and restorations.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a schematic of the invented system.

FIG. 2 is a perspective view in partial section, showing the structure of hand hold probe.

FIG. 3 is a side plan view showing the structure of scanning head in FIG. 2.

FIG. 4 is a schematic showing the layout of the scanning system.

FIG. 5 is a flowchart depicting the process to form three dimensional images.

### DETAILED DESCRIPTION OF THE PREFERRED EMBODIMENT

With reference now to the drawings, the preferred embodiment of the three-dimensional scanner is herein described. It should be noted that the articles “a”, “an”, and “the”, as used in this specification, include plural referents unless the content clearly dictates otherwise.

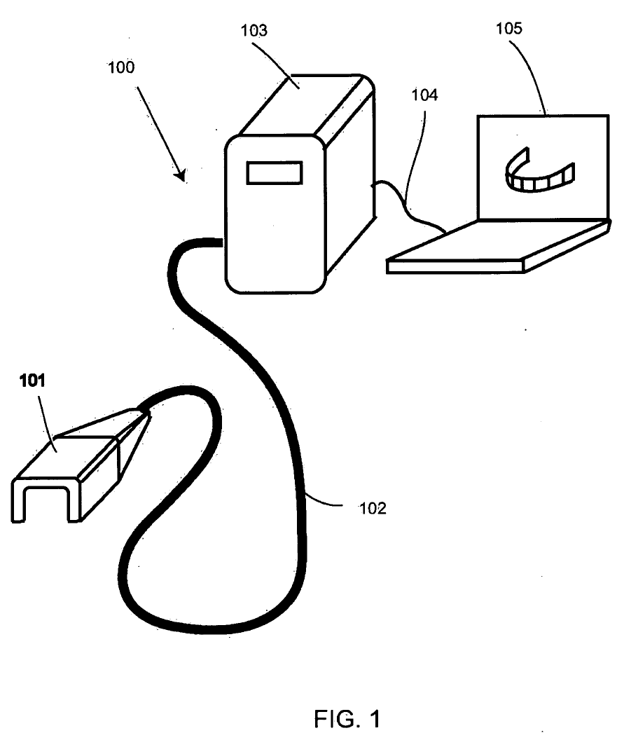

With reference to FIG. 1, 100 is the system itself while 101 is a hand probe with a three-dimensional scanning head. Cable 102 contains both optical fiber and electrical cables inside. Processing consol 103 includes controls for scanning heads, light sources, CCD cameras, and process CPU. Processing consol 103 is connected to computer 105 by cable 104. Computer 105 is provided with customized software for imaging processing. U8i

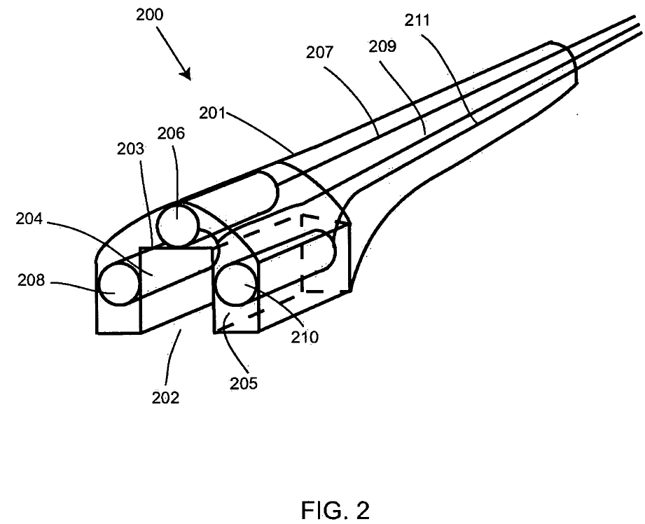

FIG. 2 depicts the hand probe (FIG. 1, 101) used in the present invention, where 200 is the hand probe. Housing 201 provides open space 202 surrounded by three walls 203, 204, and 205, respectively. The open space 202 is sufficient to allow the probe 200 to encompass the objects to be scanned, teeth as illustrated in the figures, though other scanner sizes could be used to scan other objects and use the same structures, configurations and methods as described herein to generate three-dimensional images. The walls are made of transparent materials like quartz, glass, or plastic. Inside housing 201, proximate each wall, there are three scanning heads. Scanning head 206 with a conduction cable 207 are proximate the facet of 203. Scanning head 208 with a conduction cable 209 are proximate facet 204. Scanning head 210 with a conduction cable 211 are proximate facet 205. The conduction cables 207, 209, and 211 include both optical and electrical cables and are generally contained in cable 102 in FIG. 1.

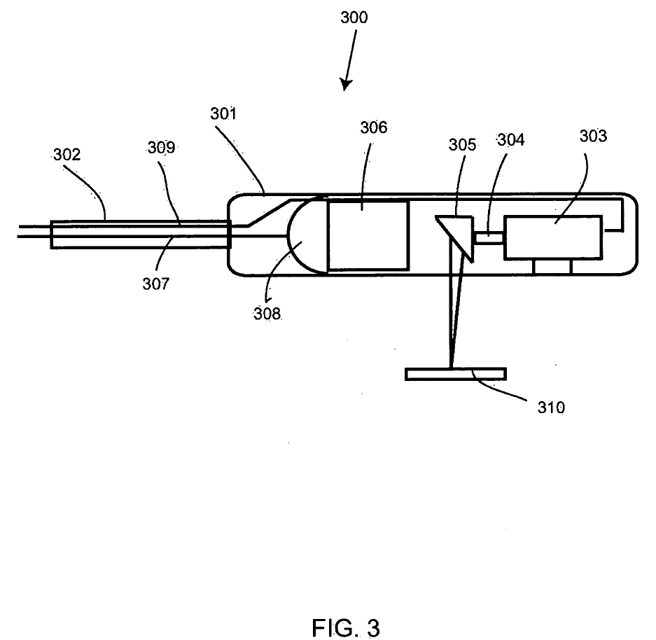

FIG. 3 depicts a scanning head 300 (206, 208 and 210 in FIG. 2). Housing 301 is made of transparent materials like quarts, glass, or plastic. Cable jacket 302 covers and protects optical fiber 307, and power cable 309. Micro-motor 303 is powered thorough power cable 309 and has a shaft 304 that can move along horizontal axis when rotating. A prism 305 is attached to the shaft so that prism can be rotated 360 degrees. A Graded index (“GRIN”) lens 306 is in front of prism 305. Optical fiber 307 is attached to GRIN lens using epoxy 308. The fiber 307 is used to transport the light source to prism 305 and transport the reflection light back to process center 103 (FIG. 1). With a prism 305 moving in horizontal direction while rotating, the prism can transfer the light from fiber to an object surface 310. The area that from which light can be collected depends on the reflection angle of prism and moving distance of prism. The light is reflected by the object surface 310, back to prism 305, collected by GRIN lens 306 and is transported through fiber 307 back to the process center 103. Thus the image on the object can be formed.

Three scanning probes, each in a different facet, can work independently to collect the surface image in different views. By using construction software, the images from different directions can be constructed to form a three-dimensional image.

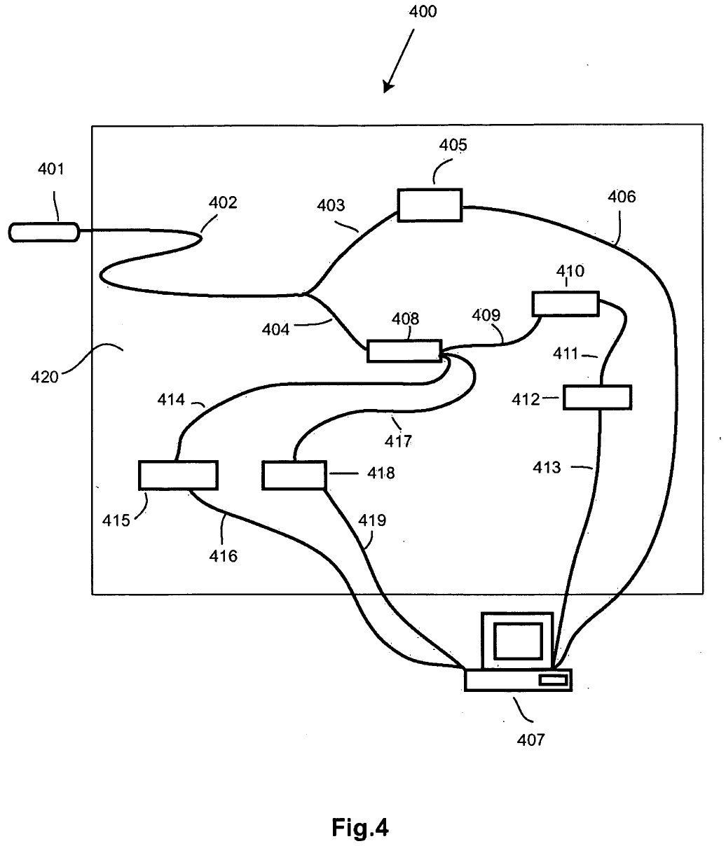

FIG. 4 illustrates to overall system 400. The three-dimensional probe 401 as illustrated in FIG. 3 is connected to cable 402, containing both electrical and optical fiber cable. Electrical cable 403 branches from cable 402 and connects to control logic circuit 405 to control the micro-motor in the probe 401. Electrical cable 406 connects control logic circuit 405 to computer 407. Fiber cable 404 also branches from cable 402 and is connected to another fiber cable 409 through coupler 408. Light source 410 is connected to fiber 409 and electrical cable 411, which connects it to its control 412. The light source can be a laser or LED, or other light sources that can be used for OCT. Electrical cable 413 connects light control 412 to computer 407. Optical fiber cable 414 extends from coupler 408 and connects to detector 415, which is used to detect return signals from the probe 401. Electrical cable 416 connects detector 415 to computer 407 for data exchange. Spectrometer 418, which is used to aid in the matching and integration of the images, is connected to computer 407 through electrical cable 41 9 and to the fiber coupler 408 through fiber cable 417. The processing console 420 (103 in FIG. 1) physically contains all of the above referenced components (at least partially) except the computer 407, and the probe 401. The electrical cables 405, 413, 416, and 419 may connect individually to the computer 407 or may be combined into one multi-cable (104 in FIG. 1).

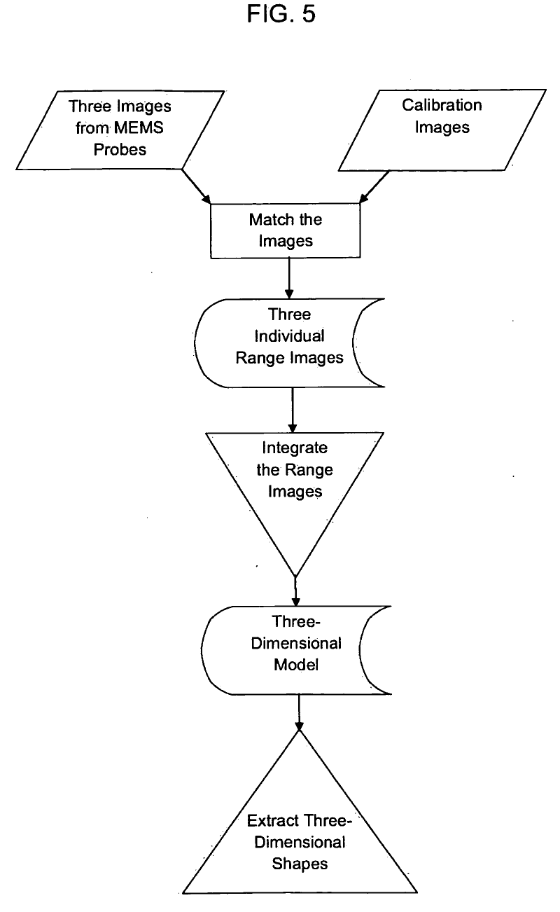

The working principle for scanning system is depicted in FIG. 5. The three images from the scanner are combined with calibration images (previously, later or contemporaneously obtained with the scanner images) and matched to yield three individual range images, each one including three-dimensional information for a surface. The range images are then integrated to form a three-dimensional model, from which three-dimensional shapes may be extracted for diagnosis.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method of producing a three-dimensional model, the method comprising:
a. generating a plurality surface images of a body to be modeled;
b. using a process to match each surface image and create range images, one of each surface imaged;
c. integrating the range images to construct a three-dimensional digital model.

2. The method of claim 1, the surface images being taken simultaneously from one scanning apparatus.

3. The method of claim 2, the surface images being at least three in number.

4. The method of claim 1, wherein OCT technology is employed to generate the images for diagnostics.

5. An imaging probe comprising:
a. a hand-piece;
b. a scanning trough, further comprised of three transparent orthogonally related walls, extending from said hand-piece;
c. three scanning heads, with one situated in each wall; and
d. primary optical and power cables to connect imaging probe to an imaging system;
Wherein the scanning heads are capable of gathering three separate views of optical data in a single pass and said data is passed to the imaging system so as to generate a three-dimensional image from the data.

6. The imaging probe of claim 4, the scanning heads each further comprising:
a. a micro-motor;
b. a shaft cantileverally extending from said micro-motor and being capable of rotational and longitudinal motion, being driven by said micro-motor;
c. a prism situated on an end of the shaft opposite the micro-motor;
d. a graded index lens situated proximate the prism, opposite the shaft;
e. optical fiber situated to receive and transmit light emitted from the prism; and
f. power and optical cable to drive the micro-motor and to inject light into the scanning head;
wherein the power and optical cables and optical fiber connect to the primary power and optical cables of the imaging probe and enable transmittal of data to the imaging system.

7. A three dimensional imaging system comprising:
a. The three-dimensional probe connected to a combined cable containing both electrical and optical fiber cable;
b. said electrical cable connected to a control logic circuit;
c. further electrical cable connecting the control logic circuit to a computer;
d. the optical fiber cable connected to a second optical fiber cable through a multi-cable coupler, the second optical fiber cable connected to a light source which is in turn electrically connected to a light control unit which is, in turn, in electrical and communicative connection with the computer.
e. a third optical fiber cable extending from the coupler and connected to a detector, which is used to detect return signals from the probe, the detector, then, electrically and communicatively connected to the computer for data exchange; and
f. a spectrometer, which is used to aid in the matching and integration of the images, connected to the fiber coupler through a fourth optical fiber cable and in operable connection with the computer;
wherein the system will collect image data and forward said data to the computer, which will generate scanned imaged to be matched into range images and will then generate a three-dimensional digital model of a scanned body.

8. The system of claim 6, said three-dimensional scanning probe further comprising;
a. a hand-piece;
b. a scanning trough, further comprised of three transparent orthogonally related walls, extending from said hand-piece; and
c. three scanning heads, with one situated in each wall.

9. The imaging system of claim 7, the scanning heads each further comprising:
a. a micro-motor;
b. a shaft cantileverally extending from said micro-motor and being capable of rotational and longitudinal motion, being driven by said micro-motor;
c. a prism situated on an end of the shaft opposite the micro-motor;
d. a graded index lens situated proximate the prism, opposite the shaft;
e. optical fiber situated to receive and transmit light emitted from the prism; and
f. power and optical cable to drive the micro-motor and to inject light into the scanning head;
wherein the power and optical cables and optical fiber connect to the combined cable of the whole imaging system and enable transmittal of data back to the consol and computer of the imaging system, the micro-motors being controlled by the control logic circuit.

10. An imaging system comprising:
a. a three dimensional scanning probe having three orthogonally related surfaces to cover a three-dimensional surface to be scanned
b. a process console containing light source and sensing devices
c. an operation system to process collected data when the surface is scanned and construct a three-dimensional image of the scanned surface.
